(19) United States
(12) Patent Application Publication
(10) Pub. No.: US 2013/0012857 A1
(43) Pub. Date: Jan. 10, 2013

(54) Wound Dressing

(75) Inventors: Nigel J. Flynn, Flowery Branch, GA (US); Harold T. Kelly, JR., Burke, VA (US); Joanne S. Walter, Blue Ridge, GA (US)

(73) Assignee: NORTH AMERICAN RESCUE, LLC, Greer, SC (US)

(21) Appl. No.: 13/635,159

(22) PCT Filed: Mar. 16, 2011
(86) PCT No.: PCT/US11/28634

§ 371 Date: Sep. 14, 2012

Related U.S. Application Data
(60) Provisional application No. 61/314,322, filed on Mar. 16, 2010.

(51) Int. Cl. A61F 13/00; B02C 23/08; A61L 15/28

(52) U.S. Cl. 602/43; 424/445; 241/24.1; 241/24.29

(57) ABSTRACT

A microfibrillar cellulose substrate having a defined morphology and optimized concentrations of free radicals formed by milling a source material containing alpha cellulose with non-metalic cutting surfaces into a sterilized fine powder with a particle size of approximately 20 to 35 micrometers. A gauze selected from the group consisting of a woven, nonwoven, and extruded netting substrate for supporting the microfibrillar cellulose substrate. The microfibrillar cellulose substrate may be carried between a first layer of the gauze and a second layer of the gauze by affixing the first and second gauze layers closed around their perimeter.

### BACKGROUND OF THE INVENTION

1) Field of the Invention

The present invention relates to a cellulosic powder for accelerated wound healing, and more particularly, to a cellulosic powder substantially free from chemical and metals contamination and having an optimized concentration of free radicals and particle morphology that promotes accelerated wound healing. In a preferred arrangement, the cellulosic powder is carried by a support structure, such as gauze, to serve as a bandage with accelerated wound healing properties.

2) Description of Related Art

According to a report produced by the US Army Institute of Surgical Research and the US Army Medical Material Development Activity, “hemorrhage from wounds remains the leading cause of mortality on the battlefield, accounting for 50% of all deaths. Hemorrhage is also the second leading cause of mortality among injured civilians, accounting for 39% of civilian trauma deaths. The primary field-ready methods for control of hemorrhage—tourniquets, direct pressure, bandages, and clamping—have not changed greatly in several centuries.”¹

Medics need to have a hemostatic agent that not only stops hemorrhaging (bleeding) in the most useful form function, but also accelerates the healing of the wound, and helps relieve the pain and suffering of the casualty. Current solutions are inadequate in addressing these issues.

Many hemostatic agents have been developed and put into use for combat medics and civilian first responders, including hemostatic agents made from chitosan, kaolin and potato starch as well as cellulose. Some of these hemostats, however, are thermogenic and increase risk to the casualty. Others have a form factor that works poorly on the battlefield under the stress and rigors of combat, or that medics find unsuitable to address the wounds that the hemostatic agent was intended to treat. Cellulose-based products that purport to have hemostatic, antimicrobial and analgesic properties are known. There are, however, no known examples of a cellulose-based product that has each of these properties and also has a concentration of free radicals and particle morphology optimized to promote wound healing. Thus, medics need better hemostatic products in a better form factor.

U.S. Pat. No. 5,667,501 entitled “Wound Dressings” identifies a polysaccharide having hydroxyl functional groups being converted into groups which are persistent free radicals or precursors to persistent free radicals. This patent claims, primarily, a wound dressing which comprises an addition polymer having hydroxyl, carbonyl or amide functional groups or a polysaccharide having hydroxy functional groups, the functional groups having been converted to derivatives which are persistent free radicals or precursors of persistent free radicals, which free radicals are reactive with molecular oxygen in a mammalian wound environment comprising macrophages and/or fibroblasts, the reaction being such as to form hydrogen peroxide in the environment; wherein the polymer stimulates activity of the macrophages or proliferation of the fibroblasts in the wound environment. The patent does not teach producing a cellulosic powder in a manner substantially free of chemical and metal contamination that results in an optimized concentration of free radicals with a defined particle morphology to increase wound healing.

A publication by Fizhin et al.² (2007) reported the results of an experiment to study the effectiveness of treating balanoposthitis patients with a deformed cellulose made from medical cotton gauze presenting as a finely dispersed powder with particle size of 20-50 micrometers (microns, or μm) purportedly free of foreign particles. This paper does not specify any particle morphology or otherwise address the effect of free radical activity as a contributor to the wound healing process or how to produce a cellulose powder substantially free from metal and chemical contamination with a defined particle morphology and a desired free radical concentration.

Further, the deformed cellulose described in Fizhin et al.² (2007) was manufactured pursuant to the process described in Russian Federation Patent RU 2,390,591 entitled “Method of Producing Cellulose Surgical Cotton”. This process is characterized by mechanical processing of cellulose cotton fibers by means of roll milling with counter-rotating rollers at a temperature of 50-200° C. and a contact time of the fibers with the rollers for 0.5-2.0 seconds, such that the roll milling is performed at an equal rotation rate in a unit of time using rollers of different diameters, or performed at different rotation rates in a unit of time using rollers of different diameters The rollers in such mills are ordinarily made of steel or other iron alloys. As a result of the exposure and processing of the cellulose with metal rollers, the cellulose becomes contaminated with metal from the rollers, which can cause undesired effects in the wound healing process. Thus, the gauze powder of Fizhin et al. was not generally free of foreign particles.

Finely divided powders made from plant cellulose are generally benign and widely used in the pharmaceutical, food and personal care industries. However, there is no known system of cellulosic powder carried by gauze or the like to render an analgesic, antimicrobial and accelerated hemostatic agent that contributes to an accelerated wound healing solution. A sensible form factor for use of a cellulosic powder in the field is necessary for medics, particularly combat medics, who work under considerable stress that can make even simple tasks exceedingly difficult.

Accordingly, it is an object of the present invention to provide a cellulosic powder for accelerated wound healing and a method for application thereof to a wound for treatment.

It is a further object of the present invention to provide a cellulosic powder with hemostatic, analgesic, and antimicrobial therapeutic benefits.

It is a further object of the present invention to provide a cellulosic powder having an optimized concentration of free radicals being operable at the wound site to promote various conditions at the wound site that accelerate healing.

It is a further object of the present invention to provide a cellulosic powder having an optimized particle morphology to promote wound healing.

It is a further object of the present invention to provide a cellulosic powder carried by a support structure to serve as a bandage with accelerated wound healing properties and other hemostatic, analgesic, and antimicrobial therapeutic properties.

It is a further object of the present invention to provide methods of manufacturing a cellulosic powder free of chemical contamination from the route to manufacture, sterilized for safe use in humans and other animals, and with a quantified absence of synthesized nanoparticles, heavy metals, or other metallic or non-metallic contamination.

### SUMMARY OF THE INVENTION

The aforementioned objectives are accomplished by providing a wound dressing comprising a microfibrillar cellulose substrate defined by a finely divided powder consisting primarily of alpha cellulose fibers processed by cutting a source material to a defined particle size; said particle size of said powder ranging from about 5 μm to 75 μm so that said particles are characterized by a high ratio of crystallinity; said particles having a generally rodform shape with deformed amorphous regions providing increased surface areas containing elevated levels of free radicals such that the concentration of free radicals on each said particle ranges from about 10⁻⁶ to 10⁻¹⁰ moles; wherein said particles form a microfribrillar matrix with optimized concentrations of free radicals that when applied to a hemorrhaging wound site provide rapid hemostatic, analgesic, antimicrobial and accelerated wound healing properties for wound care management.

In a further embodiment, the wound dressing includes a support structure carrying said microfibrillar cellulose substrate for exposure to the wound site.

In a further embodiment, the support structure consists of a gauze selected from the group consisting of a woven, nonwoven, and extruded netting substrate carrying said microfibrillar cellulose substrate.

In a further embodiment, the said microfibrillar cellulose substrate is carried between a first layer of said gauze and a second layer of said gauze by affixing said first and second gauze layers closed around their perimeter.

In a further embodiment, the source material is selected from a plant material containing approximately an 85% or higher proportion of alpha cellulose.

In a further embodiment, the source material is selected from the group consisting of cotton, wood, flax and hemp.

In a further embodiment, the source material is treated non-chemically to provide purified alpha cellulose fibers for processing into said microfibrillar cellulose substrate.

In a further embodiment, the source material is treated chemically to provide purified alpha cellulose fibers for processing into said microfibrillar cellulose substrate.

In a further embodiment, the particle size is within a range of approximately 20 μm to 35 μm.

In a further embodiment, the concentration of free radicals on each particle is within a range from about 10⁻⁶ to 10⁻⁸ moles.

In a further embodiment, the particles have a crystallinity index within a range of 0.65 to 0.80.

In a further embodiment, the moisture content and pH of said microfibrillar cellulose substrate is at standard equilibrium conditions.

In a further embodiment, the surface texture of said particles is predominantly smooth and not crenulated.

In a further embodiment, the source material is processed using a Wiley mill with a non-metallic cutting surface minimizing exposure of said source material to metallic components to obviate metal ion contamination of said microfibrillar cellulose substrate.

The aforementioned objectives are further accomplished by providing a method of preparing a microfibrillar cellulose substrate for use as a wound dressing comprising the steps of: selecting a source material containing at least an 85% proportion of alpha cellulose fibers; cutting in a shearing motion said alpha cellulose fibers into a finely divided powder so that said fibers having a generally rodform shape with deformed amorphous regions providing increased surface areas containing elevated levels of free radicals such that the concentration of free radicals on each said particle ranges from approximately 10⁻⁶ to 10⁻¹⁰ moles; sorting said finely divided powder in to ranges of particle size; selecting particles with a size ranging from about 5 μm to 75 μm so that said particles have a high ratio of crystallinity; and, purifying and sterilizing the sieved powder for application to a wound site.

In a further embodiment, the method includes the step of providing a support structure carrying said powder for exposure to the wound site.

In a further embodiment, the method includes the step of providing a gauze selected from the group consisting of a woven, nonwoven, and extruded netting substrate for supporting said powder.

In a further embodiment, the method includes the step of enclosing said powder between a first layer of said gauze and a second layer of said gauze by affixing said first and second gauze layers closed around their perimeter.

In a further embodiment, the method includes the step of cutting said source material using a non-metallic cutting surface minimize exposure of said source material to metallic components to obviate metal ion contamination of the microfibrillar cellulose substrate.

In a further embodiment, the method includes the step of selecting said source material from the group consisting of cotton, wood, flax and hemp.

In a further embodiment, the method includes the step of sorting said particles by sieving the particles through screens mill for selecting particles of a desired size.

In a further embodiment, the method includes the step of sorting said particles through an air classifying mill for selecting particles of a desired size.

### BRIEF DESCRIPTION OF THE DRAWINGS

The construction designed to carry out the invention will hereinafter be described, together with other features thereof. The invention will be more readily understood from a reading of the following specification and by reference to the accompanying drawings forming a part thereof, wherein an example of the invention is shown and wherein:

FIG. 1 shows a cellulosic powder immobilized in gauze to serve as a bandage with accelerated wound healing properties according to the present invention;

FIG. 2 shows a microfibrillar structure created by disrupting the crystallinity of the cellulose to create amorphous regions therein characterized by increased free radical active site concentration and a massively enlarged surface area of the cellulose according to the present invention;

FIG. 3 shows a schematic of high macrophage activity directed at the amorphous regions of the cellulose containing increased free radical sites for accelerated wound healing according to the present invention;

FIG. 4 shows a schematic of the healing process from an early stage with high macrophage activity to a late stage with high fibroblast activity according to the present invention;

FIG. 5 shows a schematic of high fibroblast activity at the wound site for accelerated wound healing according to the present invention;

FIG. 6 shows an electron microscope image of the cellulosic powder according to the present invention at 100 times magnification; and,

FIG. 7 shows an electron microscope image of the cellulosic powder according to the present invention at 1000 times magnification.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits and advantages of the present invention will be apparent from this summary and certain embodiments described below.

### DETAILED DESCRIPTION OF A PREFERRED EMBODIMENT

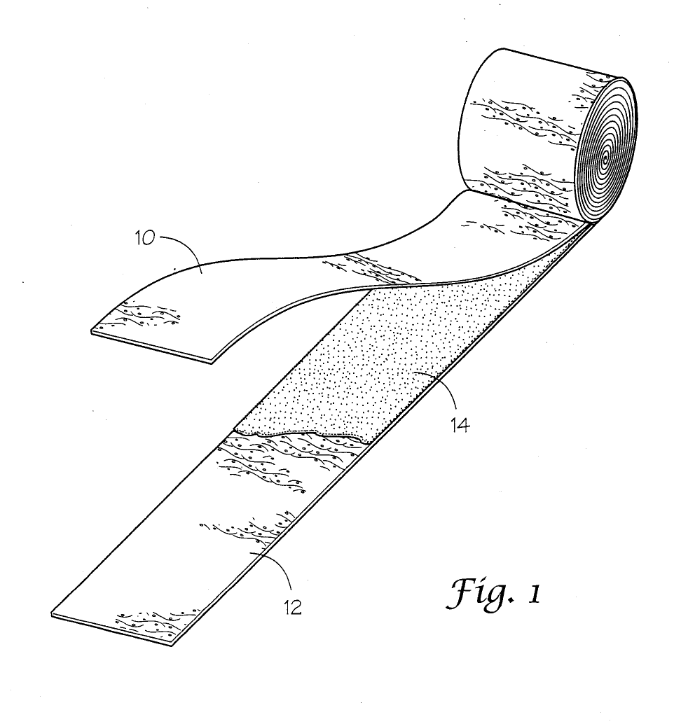

With reference to the drawings, the invention will now be described in more detail. Referring to FIG. 1, the present invention in one embodiment comprises an arrangement of a microfibrillar cellulosic substrate (“MCS”) 14 carried between an upper layer of gauze 10 and a lower layer of gauze 12 to serve as a bandage with accelerated wound healing properties. The invention uses an MCS having an increased surface concentration of free radicals and a defined particle morphology. The invention also establishes methods for deforming cellulose to optimize the particle morphology of the MCS and the concentration of free radicals with minimal metal contamination to promote the wound healing process. The present invention identifies the MCS defined herein as a compound that promotes wound healing, and sets forth the combination of this MCS compound in a support structure, such as a trauma gauze system, optimized to promote the healing process.

In one aspect of the invention, the increased surface concentration of free radicals results from a mechanical only (non-chemical) deformation of alpha cellulose into a fine cellulosic powder that alters the morphology, and thus the chemical reactivity, of the alpha cellulose. The mechanical deformation of the alpha cellulose is further controlled to produce a desired particle size found to achieve accelerated healing results. The invention offers superior product attributes for end-use application in the emergency wound care management field, which includes battlefield wound care, first response wound care, and other casualty response wound care. In particular, the cellulosic powder of the present invention exhibits a unique combination of emergency wound care and accelerated wound healing benefits including operating as a rapid hemostat to control bleeding, an analgesic providing patient pain relief without narcotics, an antimicrobial agent to resist infection, and an accelerated wound healing solution as a result of the optimized concentration of free radicals and particle morphology.

In another aspect of the invention, the cellulosic powder is defined by a free radical activated, finely divided powder substrate produced from alpha cellulose. It is axiomatic that contaminated products are inappropriate for treating wounds. Accordingly, a feature of the invention is that the microfibrillar cellulose substrate derived from the alpha cellulose is exposed to only a necessary minimum of metal during its process of manufacture to avoid metallic contamination, particularly with heavy metals, an aspect heretofore unrecognized in the prior art. A subsequent sterilization process, such as exposure to gamma radiation or other commonly known methods of sterilization by those skilled in the art, can purify or sterilize the MCS, and also confirm the absence of synthesized nanoparticles and other possible contaminants. In a further aspect of the invention, the cellulosic powder can be used alone in a powdered state or, as illustrated in a preferred embodiment of FIG. 1, can be immobilized in bandages made from various materials.

The MCS is preferably produced from plant material containing a very high proportion of alpha cellulose, such as cotton or wood. For example, the cotton can take the form of cotton linter pulp that has had its waxy solids extracted with hot water or some other extraction process to produce a high alpha cellulose content product with optimum purity. The wood can take the form of kraft pulped wood chips. Plants of the Eucalyptus genus are highly desirable for pulping because they have a very high fiber content. During the pulping process, cotton linters, wood chips or other plant fiber sources are converted into a thick fiber board which can be shipped to paper mills or other manufacturing plants for further processing. Pulp can be manufactured using mechanical, semi-chemical or fully chemical methods (kraft and sulfite processes), and the finished product may be either bleached or non-bleached.

An activation process intrinsic to the MCS produced from alpha cellulose fibers according to the methods disclosed herein is a fundamental aspect of the invention. This activation process is characterized by: 1) disrupted crystallinity of the alpha cellulose, 2) increased free radical active site concentration, 3) massively enlarged surface area of the alpha cellulose, and 4) creation of a microfibrillar structure.

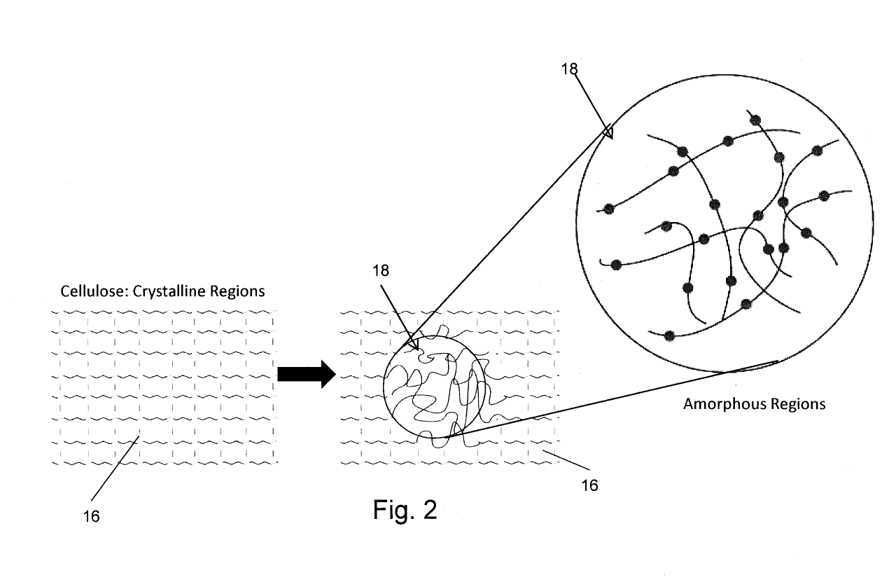

Referring to FIGS. 2, 6 and 7, to achieve these activation conditions, the particle size of the cellulose fiber substrate is reduced using a process that results in particle with high ratios of crystallinity as discussed further herein below. Moreover, the process of size reduction results in the crystalline regions 16 of the cellulose fiber particles, designated generally as 15, being deformed to produce amorphous regions, designated generally as 18, with high concentrations of free radical sites. The surface area of the cellulose fiber particles is also thereby massively enlarged through the creation of amorphous regions 18. These amorphous regions result in the generation of a “microfibrillar matrix”, a matrix of cells formed from microfibrils organized into bundles. This microfibrillar matrix is characterized by newly created free radical sites, with approximately a five-fold increase of free radical active zones. As the crystallinity of the cellulosic powder increases, so does the concentration of the free radicals present in the powder through the deformation of crystalline regions 16 into amorphous regions 18. The crystallinity of the powder increases the more its size is reduced. These free radical sites of the amorphous regions 18 are then brought into intimate contact with the wound site to result in the various benefits described herein.

The generation of the microfibrillar matrix and the creation of free radical sites that result from the manufacturing process described herein are essential to the superior healing properties of the MCS of the present invention.

The wound treatment process initiated by use of the MCS as provided herein works in two steps. The first step is a rapid hemostatic action. The term hemostatic agent (or material) is defined as any agent or material that is capable of arresting, stemming, or preventing bleeding by means other than inducing tissue growth alone. Preferably, the agent or material will be one that enhances blot clot formation. It will, of course, be appreciated that the agent or material may have the beneficial property of inducing tissue growth in addition to retarding or preventing bleeding. Examples of preferred hemostatic agents which enhance blood coagulation include carboxymethylcellulose and oxidized cellulose. Falling under the definition of “hemostatic agent” is the cellulose-based cotton gauze. The early successful application of the cotton gauze as a hemostatic construct led to the association that the fibrous structure of the gauze worked to interrupt the flow of blood by initiating the clotting cascade through contact activation. This, in turn, provided the incentive to examine other fibrous constructs made primarily from natural materials, such as collagen, chitosan, and alginate, which led subsequently to the discovery of surface charge contribution to hemostasis known in the art.

The MCS is applied, either directly in powder form or as contained within a bandage, to an open and bleeding wound surface in a quantity sufficient to cause a rapid concatenation of the finely divided particles and a rapid occlusion of the wound site resulting in hemostasis. Several hours after application the MCS should be irrigated and replaced by sterile MCS sufficient to uniformly cover the wound, which should be done at regular intervals to ensure that the therapeutic benefits provided by the second step of the healing process can be achieved.

The mechanism of action for the hemostasis is the initiation of the clotting cascade through contact activation, or the formation of a fibrous construct in the form of a microfibrillar matrix such that red blood cells, which ordinarily have a size of 6 μm-8 μm, are attracted to and bind with the fibrous construct. It has been well established that fibrous constructs, as in the cellulose-based cotton gauzes, can aid the process of hemostasis through interrupting the blood flow and subsequent coagulation. Positively charged cellulosic fibers, such as those based on chitosan, were later recognized in the art as effective hemostatic constructs. Whether the result of a principally chemical clotting cascade process, a principally mechanical formation of a microfibrillar construct, some other process principally related to the changed morphology of the cellulosic raw material, or a combination thereof, bleeding is dramatically reduced. The exact mechanism has yet to be determined in the art. Nevertheless, for tissue engineering, it has been recognized that components of biocompatible scaffolds or matrices or nanometer or micrometer diameter fibers provide favorable environments for cell adhesion, cell proliferation, and directed cellular growth.

The second step is a healing process that gives a unique combination of analgesic and antibiotic effects. In this second step the introduction of free radicals influence two distinct stages of the healing process.

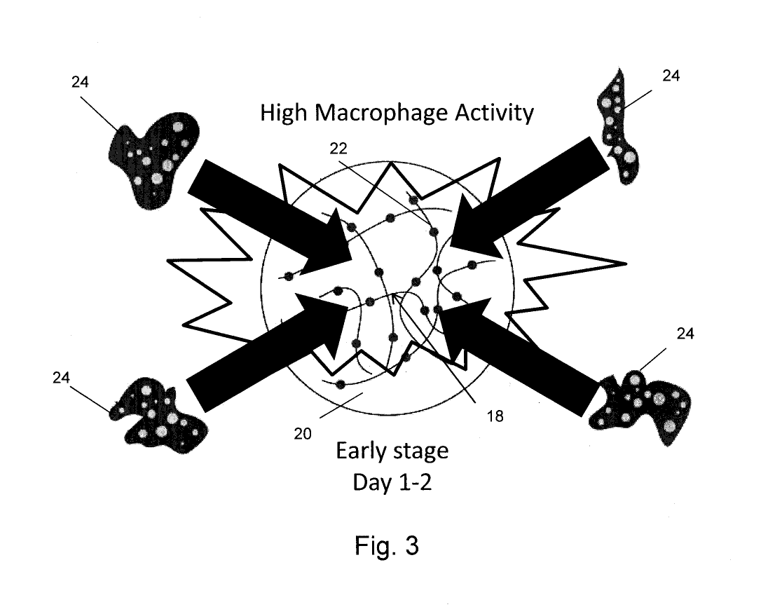

Referring to FIGS. 3-5, in the early stage of the wound healing process (first to second days), the alpha cellulose powder (MCS) leads to the formation of covalent bonds between bacteria and the free radicals present in the microfibrillar matrix of the amorphous regions 18 of the cellulose, immobilizing them, preventing reproduction, and preventing their entry into the body. The result of these processes is the creation of a surface active microfibrillar matrix that attaches to tissue 20 at the wound site and has high concentrations of free radicals 22 that operate to stop bacteria and increase macrophage 24 activity which kills bacteria.

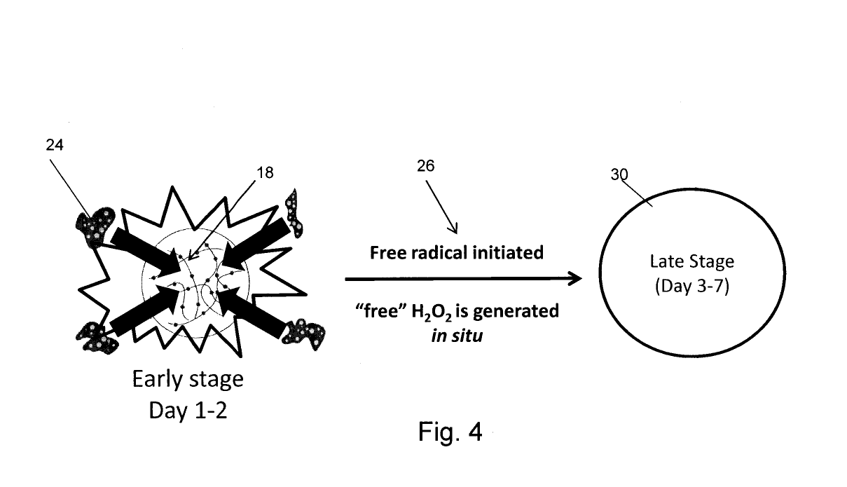

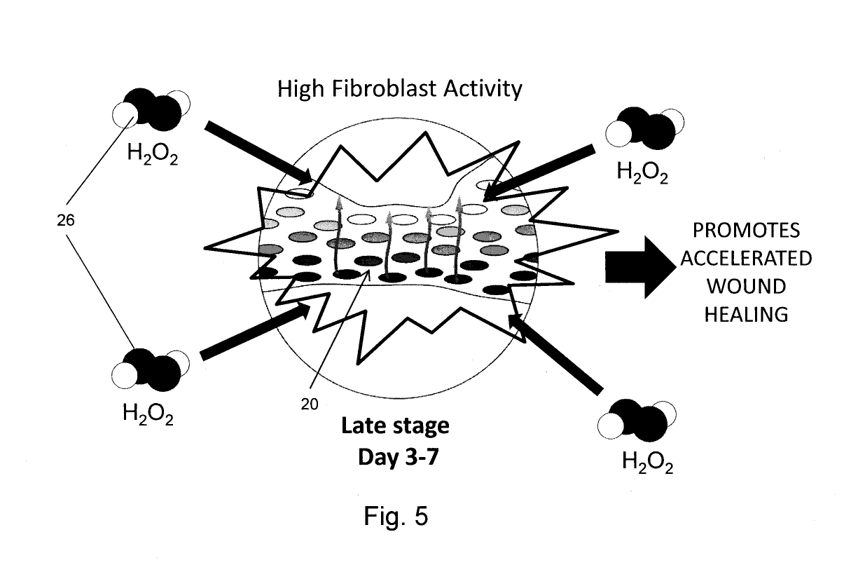

Application of the MCS to the wound site causes high macrophage activity, as noted above, during this early stage. Free radicals 22 present in the microfibrillar matrix of amorphous regions 18 attract macrophages 24 and promote a “respiratory burst”. The macrophage activity kills bacteria, removes cell debris and retards fibroblast activity. Simultaneously, free radical initiated “free” hydrogen peroxide (H₂O₂) 26 (FIGS. 4 and 5) is generated in situ, which accelerates tissue development and wound healing while numbing pain in that area.

It is known that free radicals are detrimental to late-stage healing 30 (FIG. 4) (third to seventh days), during which free radicals promote continued inflammation, thereby delaying healing. However, the presence of low concentrations (10⁻⁶-10⁻⁸ moles) of H₂O₂ stimulates fibroblast activity. This high fibroblast activity during the late stage 30 (FIG. 5) of the wound healing process promotes accelerated wound healing.

Although H₂O₂ is well known to disinfect wounds at a strength of 3%, application of H₂O₂ at such strength may hurt the process of tissue regeneration. As described by Roy et al. (2006)³, however, the body naturally produces low levels of H₂O₂ at the wound site, and at such micromolar levels H₂O₂ favorably influences wound angiogenesis. The presence at the wound site of H₂O₂ in such concentrations keeps the wound free from infection by promoting respiratory bursts from the neutrophils, and later stimulates the macrophages to release vascular endothelial growth factor (VEGF), an angiogenic factor crucial for wound healing. The amount of free radicals in the MCS produces a similar concentration of H₂O₂, a concentration low enough to have antimicrobial properties and thereby accelerate wound healing but not so high as to damage tissue.

The concentration of free radicals in a sample of cellulosic powder can be determined using electron paramagnetic resonance spectroscopy (EPR). This method exposes samples to microwaves that excite the spins of the unpaired electrons present in free radicals. There is a net absorption of energy, which is converted into a spectrum. Using the information found on the spectrum, calculations can be made to determine the g-value, spins/g, spins/glucose unit, and glucose units/spin. By calculating the g-value and comparing it to published g-values, one can begin to evaluate the nature of free radical concentration. Calculating the number of spins per glucose molecule translates into a calculation to determine the amount of unpaired electrons. Because free radicals have an unpaired electron, it is reasonable to equate that spins/glucose unit is equal to the free radical concentration. In a preferred embodiment of the invention, the MCS has a free radical concentration of 10⁻⁶-10⁻⁸ moles at the wound site.

The process of manufacture for the microfibrillar cellulose substrate optimizes surface free radical concentration and particle morphology, which improves the efficacy of the cellulosic powder product. The cellulosic powder resulting from the process of manufacture comprises a free radical activated MCS with optimized characteristics. The process of manufacture of this cellulosic powder and the incorporation of the MCS into a support structure for application to a wound site are fundamental aspects of the system of the invention to achieve the desired wound care results not presently achievable in the prior art.

The process of manufacturing the microfibrillar cellulose substrate involves particular milling techniques to reduce the particle size of the alpha cellulose and deform the material to produce the disrupted crystallinity of the cellulose that causes the formation of amorphous regions 18 that define a microfibrillar matrix.

Mechanical techniques used to reduce particle size fall into one of four types: shear, impact, attrition and compression. Shearing (cutting) techniques grind material through friction by applying two forces that are equal in magnitude and opposite in direction and that act along parallel lines. Shearing techniques are useful for the size reduction of cellulose because of the fibrous structure intrinsic to cellulose and are the preferred method of processing the alpha cellulose source material to result in the desired characteristics described herein.

Impact techniques hit material against hammers or other media such as stainless steel or ceramic balls, or against pieces of the material itself. Mills that apply impact techniques include pulverizers, hammermills, pebble mills, ball mills, jet mills and air classifying mills. “Air classifying mill” is a general term for many types of impact mills that also incorporate dynamic classifier technology in one machine. The feed material enters the mill, size is reduced by impact hammers, and the reduced material is presented to the classifier mill. The product stays in the pulverization chamber until it is light enough to flow out of the mill in an air stream.

Another method of particle size reduction is accomplished in a machine referred to as a spray dryer. This machine pushes heated air or other gas onto an atomized, sprayed liquid stream within a drying chamber to cause evaporation and produce a free flowing dry powder with a controlled average particle size.

Preferably, to arrive at the preferred morphology of the particulate used in the MCS of the present invention, the process of manufacture applies the shearing technique expressed in the machine commonly referred to as a “Wiley mill”. A Wiley mill is a cutting machine that typically has two stationary hard tool steel blades and a rotor with four cutting edges that revolve at high speed. The material to be milled is placed into a hopper, and gravity drops it into the path of the blades. The revolving cutting edges work against the stationary edges to create a powder, which is forced through a steel screen. A screen discharge keeps the product in the shearing chamber until it is small enough to fall through the screen. The shearing process breaks down the longer fibers of the cellulose, increasing the number of crystalline regions while forming a rod form, needle like particle with the free radical enhanced amorphous regions 18. Preferably, the mill is equipped with non-metallic cutting surfaces to obviate metal ion contamination.

Providing an uncontaminated cellulosic powder is another important aspect of the present invention. In a preferred embodiment, because the cellulose is processed using a Wiley mill, as opposed to using a roller mill as in the prior art, the cellulose is exposed to very little actual metal contact to being with, thus reducing potential metallic contamination. As a result, the present invention provides a method of manufacture which lessens contaminants to produce a product with improved purity and sterilization relative to the prior art.

The toxicity of metals is a medically complex subject. Some heavy metals, such as lead, mercury and cadmium, have no known benefit for human physiology. Aluminum, though not a heavy metal, has been scrutinized at great length because its low atomic weight enables it to pass through the blood/brain barrier. Even trace amounts of nickel and chromium should be avoided, but trace quantities of other metals, such as zinc, cobalt and iron, are essential to human biochemical processes. Iron and aluminum are essential to human biochemical processes and should not be administered to the body except through the dietary advice of a medical doctor or dietician. Accordingly, the route to manufacture should include purification and sterilization of the microfibrillar cellulose substrate.

Concerning the novel combination of cellulosic powder and bandage material, in a further embodiment, the microfibrillar cellulose substrate can be immobilized in a nonwoven gauze for emergency wound care management. In non-emergency (such as hospital patient) wound care management, the microfibrillar cellulose substrate can alternatively be used as a powder or as expressed in other forms, including a nonwoven gauze, a powder-filled pillow or an extruded microfiber, to name just a few examples.

Accordingly, the invention includes the manufacture of a microfibrillar cellulose substrate powder subjected to a purification or sterilization process, and gauze or other mesh material suitable for carrying the powder for application to a wound site. Suitability of such material is determined by, inter a/ia, the basis weight of the substrate to be immobilized, the composition of the fibers of the material, the diameter of the fibers, the size of the aperture of the material, the concentration of the powder on the material and the uniformity thereof, and an absence of contamination of the material. The microfibrillar cellulose powder can then be immobilized in the gauze by sewing or other method. In one embodiment, the gauze container is preferably an “easy-open” packaging perforated on four sides for rapid access. Applying the gauze with immobilized powder to an open wound provides the same therapeutic benefits obtained by application of the powder without gauze, but in a form factor that will be easier for medics to use, particularly during combat.

The substrate made from alpha cellulose may be used for trauma care and wound dressing in animals, including humans and other mammals, comprising a substrate which is applied to the wound. The substrate, which is a microfibrillar cellulose substrate in the form of a powder, may be applied to the wound area as a free flowing powder of the particles of the substrate, a dry spray of particles, a moist spray or aerosol of the particles, as an association of particles in or on a support structure (such as a web, tape, fabric, foam, reticulated foam, or film), and may optionally contain other material with the particles to facilitate wound healing.

The preferred version for immobilizing the cellulosic powder in gauze is as follows:

In the most basic form function, the application of the cellulosic powder should allow for direct contact of the particles with the wound, preferably without any intermediate film or material between the wound and the powder.

The particles may generally have a size from about 5 μm to 75 μm. The particles are reduced to this size using one or more pieces of particle size reduction equipment, notably a Wiley mill. The material to be reduced with such equipment is comprised of alpha cellulose. Examples of specific material containing alpha cellulose useful in the practice of the present invention comprise plants with naturally high content of alpha cellulose (>85%) including cotton, wood, flax and hemp.

Preferred materials are non-toxic and are provided as a sterile supply.

One embodiment of this invention is characterized by the use of the cotton plant. Cotton is the plant with the highest content of alpha cellulose (>99%). The material used in this embodiment is cotton cellulose that has been extracted through a non-chemical treatment, ordinarily using hot water, of the waxy solids frequently found in cotton linters as a minor constituent, thereby avoiding possible contamination issues with other chemical and mechanical processing methods.

Another embodiment of the invention is characterized by the use of wood. Wood is appealing because of its availability and generally lower cost compared to cotton. The material used in this alternative practice is fluff pulp, a holocellulose, which is a term used to describe a combination of alpha cellulose and hemicellulose. Fluff pulps can be bleached or non-bleached, and have controlled sheet physical characteristics, which enable these pulps to be defiberized and used as granular feed to be further reduced in size. Contemporary production of fluff fibers usually avoids the use of chlorines even if the product is bleached.

Fluff pulps can be made from softwood trees and hardwood trees. A suitable form of fluff pulp is produced from wood of the eucalyptus tree, a hardwood tree with high fiber yield. This pulp product is commercially available and is sold by companies such as Suzano Pulp and Paper. The Suzano pulp is supplied in sheets measuring generally 92 cm by 67 cm and usually packaged in 250 kg bales.

Pulp can be manufactured using mechanical, semi-chemical or fully chemical methods (kraft and sulfite processes). During the pulping process the bulk structure of the plant fiber source is broken down into its constituent fibers. A pulp mill is a manufacturing facility that converts wood chips or other plant fiber source into a thick fiber board.

Wood and other plant materials used to make pulp contain three main components (apart from water): cellulose fibers, lignin (a three-dimensional polymer that binds the cellulose fibers together) and hemicelluloses (shorter-branched carbohydrate polymers). The pulping process degrades the lignin and hemicellulose into small, water-soluble molecules that can be washed away from the cellulose fibers without depolymerizing the cellulose fibers.

The finished product, which may be either bleached or non-bleached, can be sent to a mill or elsewhere for further processing. The combination of pulping and bleaching the fibers results in bright fibers with little residual lignin. Specialty pulps or fibers are fibers that are pulped using a prehydrolyzed alkaline or a conventional alkaline or acid process and bleached, then chemically modified to tailor the fibers for specific applications. The most common form of chemical modification involves purifying the fiber so that it is composed of almost pure cellulose.

The prior art identifies many different processes for making powdered cellulose and its derivatives, among them microcrystalline cellulose and microfibrillar cellulose, each of which subjects cellulose, preferably in a form whose plant origin is trees, to various wet-based chemical processes to produce a finely divided cellulosic powder. These processes include subjecting cellulose to acid hydrolysis or oxidative degradation with alkali, subjecting cellulose to steam treatment, soaking cellulose in an aqueous alkali metal hydroxide solution followed by regeneration, and subjecting wood pulp to alkali hydrolysis then acid hydrolysis. These different wet and chemical processes cleave the amorphous regions of the cellulose fibers so as to curtail or eliminate the possibility of initiating the clotting cascade through contact activation, initiating the formation of a matrix or fibrillar construct, or generating in situ an appropriate concentration of free radicals to promote an antimicrobial or analgesic response.

In a preferred embodiment of the present invention, wood is used to make a finely divided cellulose powder that, with respect to free radical concentration and particle morphology, applies a process of manufacture that is conceived and intended to be used for accelerated wound healing and not intended to be used as a biologically inert particle. The use of a dry and specific cutting/shearing mechanical process to reduce the size of wood-derived cellulose instead of a wet and chemical process, or other mechanical process, has produces synergistic results for use in wound care. Thus, the object of using such a process in a system that provides an accelerated wound healing solution has not been accomplished previously in the prior art. The features of the present invention that comparably favor relative to the prior art in this regard include superior particle morphology, increased generation of free radicals, lower cost of raw materials, and relatively low contaminating materials.

A preferred embodiment of the invention as it relates to the manufacture of the MCS begins with a sheet of delignified wood pulp, such as the Suzano bleached kraft eucalyptus pulp, which has a thickness of 2.0-2.5 mm. The sheets, which ordinarily measure 92 cm by 67 cm, are sliced with a cutting tool, such as a sharp blade, to permit an efficient introduction of the pulp into the feeder of a Wiley mill. The feed is milled by the shearing action of the blades in the Wiley mill to reduce the size of the particles of the feed and produce a powder. Gravity pulls the powder through a screen for sorting and collecting particles of a predetermined size into a container positioned at the exit of the mill. The powder is passed through a 3-inch, 8-inch, or 12-inch sieve apparatus, with the 8-inch sieve being the preferred tool. The sieve is fitted with at least two screens and a catch pan, which produces particles of three general sizes, descending as a function of the size of the apertures of the screen, the last of such screens sized to pass particles no larger than 38 μm, the smallest commonly available size. Particles of powder that do not successfully pass through the first screen can be re-fed in the Wiley mill for further milling and sieving. This combination of sieve and screen is used to collect particles that would generally not be larger than 38 μm with the mode of a given sample for use in the present invention ranging from approximately 20 μm to 35 μm, but preferably having a particle size of about 20 μm.

Use of a Wiley mill as the principal milling tool is preferred because the shearing techniques of this mill produced a finely divided cellulosic powder with a desired particle morphology as described herein. As an alternative to using a sieve with a 38 μm screen to sort particles for selection, the principles of the classifying component of an air classifying mill can be applied to the practice of this invention to classify the particles according to size. Classifying particle size using a mill of this sort, or a cyclone method applying the same principle, would involve keeping the milled powder agitated while using air, instead of gravity, to pull the powder through the screens. The advantage of using a cyclone method is a greater yield of reduced-size particles because there is no screen to which the particles could cling during sieving.

Because the substrate is primarily designated for trauma care it must be manufactured in a manner that permits its use in the body. To accomplish this the preferred method of manufacture applies a general regime of good laboratory practice and clinical good manufacturing practice emphasizing cleanliness and sterility.

Preferred functional requirements for one embodiment of the MCS manufactured according to the processes set forth herein include: 1) a finely divided powder within a preferred range of 5 μm-75 μm, preferably with a particle size of 20 μm; 2) the morphology of the particles of the MCS is characterized by a rodform shape, wherein most particles in a sample will be needle-like, not spherical, with a high aspect ratio that leads to a large specific surface within a preferred range of 3:1 (three units long, one unit wide) to 7:1, preferably with an aspect ratio of 5:1; 3) the morphology is characterized by high crystallinity, with a ratio of crystalline regions of the alpha cellulose to amorphous regions within a preferred range of 3:1 to 7:1, such that the concentration of free radicals on each particle lies within the range of 10⁻⁶-10⁻¹⁰ moles, and preferably range from 10⁻⁶-10⁻⁸; 4) the process of manufacture of the MCS has controlled metallic contamination; 5) upon completing the process of manufacture the MCS is purified and sterilized; 6) the surface texture of the particles of the MCS is predominantly smooth, not crenulated; 7) the moisture and pH of the MCS are at standard equilibrium conditions.

Samples of the MCS were produced from alpha cellulose in the form of cotton linters and holocellulose in the form of bleached kraft eucalyptus pulp using the process of manufacture described herein through non-chemical treatment on a Wiley mill. The particles of the MCS sample were evaluated to determine the particle size distribution of the sample. The evaluation was done using the Saturn DigiSizer II 5205 laser diffraction tool using isoproponal analysis.

| | | | | 10th | 50th | 90th |
|---|---|---|---|---|---|---|
| | Mean | Median | Mode | percentile | percentile | percentile |
| | diameter | diameter | diameter | diameter | diameter | diameter |
| | (μm) | (μm) | (μm) | (μm) | (μm) | (μm) |
| Cotton | 39.948 | 31.054 | 31.608 | 5.241 | 31.054 | 80.038 |
| Eucalyptus | 46.915 | 31.564 | 31.608 | 8.439 | 31.564 | 116.708 |

An analysis of the resulting data showed that the process of manufacture produced a particle that has a median range of approximately 31 μm, within the preferred range. Further processing can be undertake to achieve a preferred particle size of approximately 20 μm. The data also indicate that the process of manufacture, as applied to each form of cellulosic raw material, produces a particle that comports with what would be expected of a finely divided cellulosic substrate.

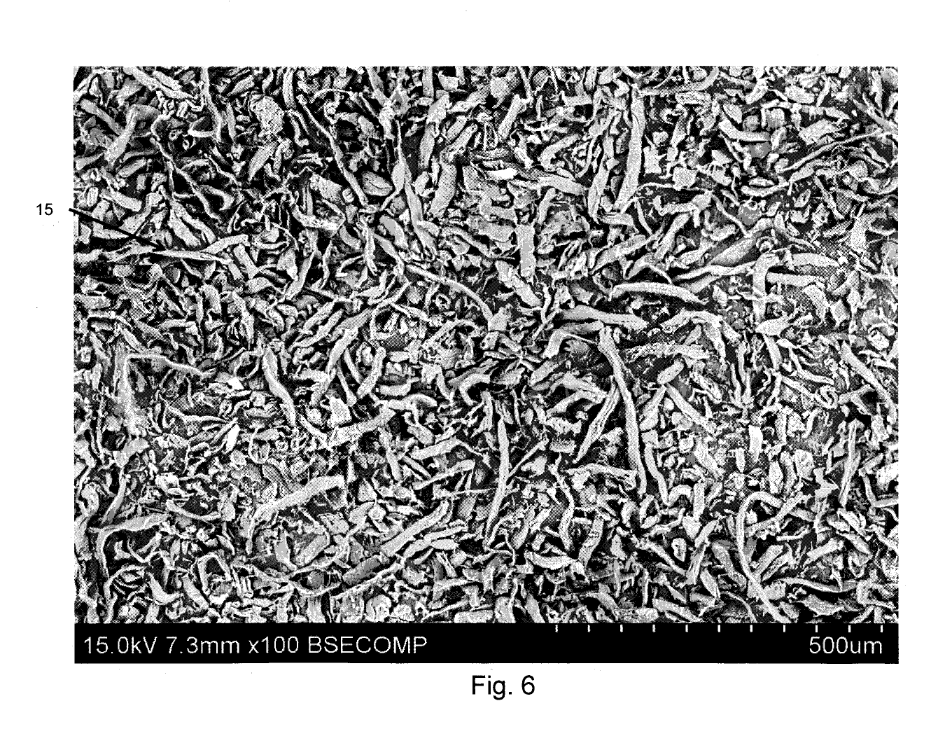

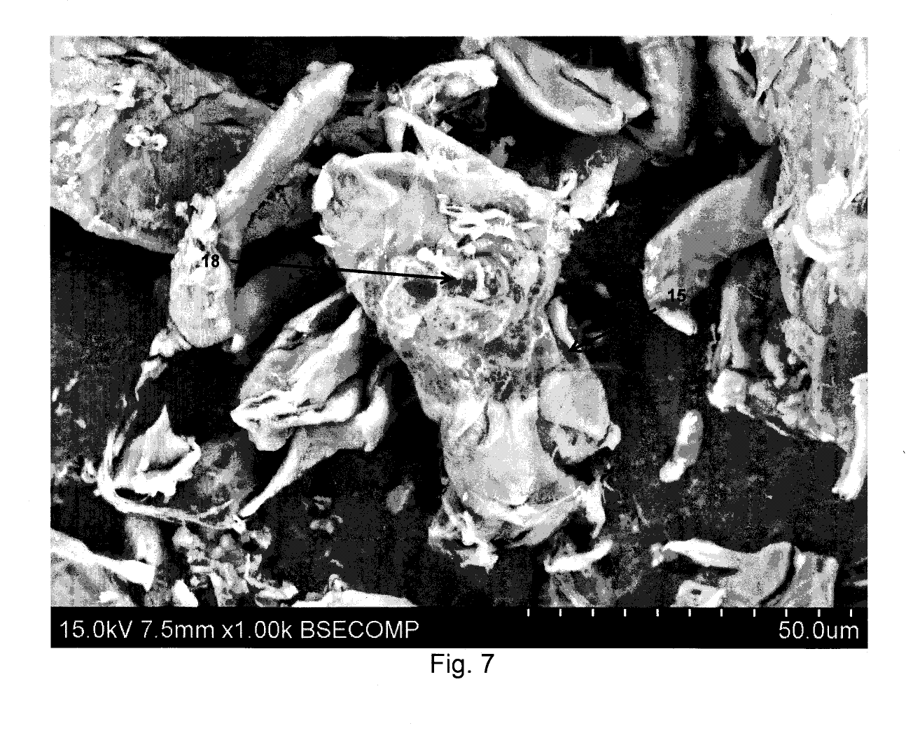

The particle morphology was evaluated with a scanning electron microscope at 100×, 200×, 1000× and 5000× magnification. As illustrated by FIGS. 6 and 7, the images produced by this microscopy analysis showed particles 15 that are rodform, fibrillar, with amorphous regions 18, and are thus desirable for hemostasis.

The crystallinity of the particles, and also the crystallinity of the raw materials in their form before milling, were analyzed using x-ray diffraction techniques. Measurements were taken a with copper x-ray source equipped in a panalytical MPD system with Bragg-Brentano geometry. An analysis of the resulting crystallography images showed that alpha cellulose is more crystalline than holocellulose, as would be expected. The milling process of manufacture of the present invention was demonstrated to have the effect of increasing the crystallinity of each of the samples tested to produce fibers with a high ratio of crystallinity. The crystallinities of the samples were found to have a crystallinity index of approximately 0.78 (78%), within a desirable range of 0.65 to 0.80 (65%-80%). The milled reference sample, a cotton ball, has a standard crystallinity index of is 0.59 (59%).

The concentration of free radicals on each sample of the particles, and also a unmilled reference sample of each raw material used to produce the samples, was evaluated using electron paramagnetic resonance spectroscopy. EPR was done at 1 mW, 24 degrees C., and 5 μl of 1 mM TEMPO was used as an integration standard. The data from this analysis is summarized in the following table:

| | moles × |
|---|---|
| | spins/gram of |
| | material |
| Cotton | 0.180 × 10\hat{ }−8 |
| Eucalyptus | 0.078 × 10 \hat{ }−8 mol/g |
| Cotton | 0.024 × 10 \hat{ }−8 mol/g |
| (unmilled) | |
| Eucalyptus | 0.026 × 10 \hat{ }−8 mol/g |
| (unmilled) | |

EPR testing revealed that the concentration of free radicals increased significantly as a result of milling each of the two MCS samples. In comparison with the control references, the spins per material ratio had seven times more spins than the unmilled reference samples. This number correlates with free radical concentration.

The sample of the MCS was evaluated using quantitative chemical analysis to determine the concentration of metallic elements in the sample. The analysis was comprised of the ICP EPA 6010 test for thirty common metals and the Total EPA 7471 Solid test for mercury.

| | Alpha | |
|---|---|---|
| | cellulose | Holocellulose |
| Element | (mg/kg) | (mg/kg) |
| Aluminum | 22.2 | 25.2 |
| Barium | 1.2 | 1.3 |
| Cadmium | ND | ND |
| Calcium | 264.0 | 80.1 |
| Chromium | 19.2 | 12.9 |
| Iron | 173.0 | 138.0 |
| Lead | ND | ND |
| Magnesium | 42.7 | 128.0 |
| Phosphorus | ND | ND |
| Silicon | 28.8 | 49.1 |
| Sodium | 183.0 | 1320.0 |
| Strontium | ND | ND |
| Zinc | 64.6 | 15.4 |
| Mercury | ND | ND |

As anticipated, iron was detected in a high concentration in the samples because the blades of the Wiley mill that was used to mill the samples were not rendered with stainless steel, ceramic, or some other blade assembly cut/grind equipment that is not contaminated by iron.

Each of the samples made from alpha cellulose and holocellulose was further examined to evaluate its behavior as finely divided powder. A standard sorbency test was performed using water. The sample made from alpha cellulose absorbs 4.82 times its own weight in water, and the sample made from holocellulose absorbs 8.85 times its own weight in water. A physical reaction test indicated that each sample exhibited non-Newtonian behavior, which is typified by a powder acting as a solid in the presence of moisture when subjected to pressure and acting as a slurry in aqueous solution with a high solids content. These tests, and information generally known about the behavior of cellulosic powders, confirm the suitability of each of the samples as a hemostatic agent.

At a minimum the MCS has been shown to be a sorbent with a sorbency ratio higher than the sorbency ratio of cellulose or cellulosic powder that has not had its surface area increased by one or more milling techniques; furthermore, since the MCS is a more efficacious sorbent it stands to reason that the MCS has hemostatic properties.

It has been learned that an alternative approach to the preparation of a MCS with the following properties: 1) morphology (including rodform shape), yielding hemostatic, analgesic, antimicrobial and accelerated wound healing properties, 2) particle size distribution, yielding accelerated wound healing, 3) free radical generation and preservation close to target range, yielding analgesic and antimicrobial activity, 4) high sorbency and non-Newtonian behavior, yielding hemostatic performance, and 5) low metal ion contamination; can be achieved through dry non-chemical processing of alpha cellulose fibers using only mechanical cutting (like a Wiley mill). The equipment when rendered with non-metallic cut/grind surfaces will obviate the metal ion contamination observed in the evaluation of the MCS samples discussed herein.

In use, the particles that comprise the powdered microfibrillar cellulose substrate, having the defined morphology, may be directly applied to wound surfaces or held in place by pressure. The powder may be free flowing or be carried in or on a support structure. For example, the powder may be adhered to a support structure that comprises a sheet or film which is applied (e.g., contacted, wrapped, adhered, secured, affixed or otherwise placed into a position where blood on the wound area will be absorbed or adsorbed by the substrate) to a wound. The powder may also be provided in a form where it may be interspersed with fibers, filaments or other particles in a self-supporting structure, entangled within the fibrous elements of a net, web, fabric or sheet, embedded in a sheet or film (with the particles exposed to enable adsorption or absorption of blood in contact with the wound), or a packet of material. The powder may also be provided as part of a patch system, with a fibrous network associated with the substrate to provide a high level of structural integrity and strength to the applied assembly over the wound.

Referring to FIG. 1, in the illustrated embodiment, the cellulosic powder 14 is carried by a gauze support structure by affixing, for example by sewing, the gauze closed around its perimeter with the powder 14 disposed between two layers of gauze 10 and 12. The gauze is delivered to the user in packaging that is perforated on all four sides to facilitate easy tearing with hands or teeth. The gauze may be comprised of a woven, nonwoven, extruded netting or other appropriate substrate capable of supporting the cellulosic powder.

While a preferred embodiment of the invention has been described using specific terms, such description is for illustrative purposes only, and it is to be understood that changes and variations may be made without departing from the spirit or scope of the following claims.

### REFERENCES

1. Kheirabadi et al., “Development of Hemostatic Dressings for Use in Military Operations” (2004). http://handle.dtic.mil/100.2/ADA444927

2. Vladimir Fizhin and Dmitry Fizhin, Kazan State Medical University, Second All-Russian Congress of Dermatologists (St. Petersburg, Sep. 25-28, 2007). www.dermatology.ru/collections/tseloform-v-lechenii-balanopostita.

3. Roy et al., “Dermal Wound Healing in Subject to Redox Control,” Molecular Therapy, Vol. 13, No. 1, January 2006, p. 211.

What is claimed is:

1. A wound dressing comprising:
a microfibrillar cellulose substrate defined by a finely divided powder consisting primarily of alpha cellulose fibers processed by cutting a source material to a defined particle size;
said particle size of said powder ranging from about 5 μm to 75 μm so that said particles are characterized by a high ratio of crystallinity;
said particles having a generally rodform shape with deformed amorphous regions providing increased surface areas containing elevated levels of free radicals such that the concentration of free radicals on each said particle ranges from about 10⁻⁶ to 10⁻¹⁰ moles;
wherein said particles form a microfribrillar matrix with optimized concentrations of free radicals that when applied to a hemorrhaging wound site provide rapid hemostatic, analgesic, antimicrobial and accelerated wound healing properties for wound care management.

2. The wound dressing of claim 1, including a support structure carrying said microfibrillar cellulose substrate for exposure to the wound site.

3. The wound dressing of claim 2, wherein said support structure consists of a gauze selected from the group consisting of a woven, nonwoven, and extruded netting substrate carrying said microfibrillar cellulose substrate.

4. The wound dressing of claim 3, wherein said microfibrillar cellulose substrate is carried between a first layer of said gauze and a second layer of said gauze by affixing said first and second gauze layers closed around their perimeter.

5. The wound dressing of claim 1, wherein said source material is selected from a plant material containing approximately an 85% or higher proportion of alpha cellulose.

6. The wound dressing of claim 1, wherein said source material is selected from the group consisting of cotton, wood, flax and hemp.

7. The wound dressing of claim 1, wherein said source material is treated non-chemically to provide purified alpha cellulose fibers for processing into said microfibrillar cellulose substrate.

8. The wound dressing of claim 1, wherein said source material is treated chemically to provide purified alpha cellulose fibers for processing into said microfibrillar cellulose substrate.

9. The wound dressing of claim 1, wherein said particle size is within a range of approximately 20 μm to 35 μm.

10. The wound dressing of claim 1, wherein said concentration of free radicals on each said particle is within a range from about 10⁻⁶ to 10⁻⁸ moles.

11. The wound dressing of claim 1, wherein said particles have a crystallinity index within a range of 0.65 to 0.80.

12. The wound dressing of claim 1, wherein the moisture content and pH of said microfibrillar cellulose substrate is at standard equilibrium conditions.

13. The wound dressing of claim 1, wherein a surface texture of said particles is predominantly smooth and not crenulated.

14. The wound dressing of claim 1, wherein said source material is processed using a Wiley mill with a non-metallic cutting surface for minimizing exposure of said source material to metallic components to obviate metal ion contamination of said microfibrillar cellulose substrate.

15. A method of preparing a microfibrillar cellulose substrate for use as a wound dressing comprising the steps of:
selecting a source material containing at least an 85% proportion of alpha cellulose fibers;
cutting in a shearing motion said alpha cellulose fibers into a finely divided powder so that said fibers having a generally rodform shape with deformed amorphous regions providing increased surface areas containing elevated levels of free radicals such that the concentration of free radicals on each said particle ranges from about 10⁻⁶ to 10⁻¹⁰ moles;
sorting said finely divided powder in to ranges of particle size;
selecting particles with a size ranging from about 5 μm to 75 μm so that said particles have a high ratio of crystallinity; and,
purifying and sterilizing the sieved powder for application to a wound site.

16. The method of claim 15, including the step of providing a support structure carrying said powder for exposure to the wound site.

17. The method of claim 15, including the step of providing a gauze selected from the group consisting of a woven, nonwoven, and extruded netting substrate for supporting said powder.

18. The method of claim 17, including the step of enclosing said powder between a first layer of said gauze and a second layer of said gauze by affixing said first and second gauze layers closed around their perimeter.

19. The method of claim 15, including the step of cutting said source material using a non-metallic cutting surface for minimizing exposure of said source material to metallic components to obviate metal ion contamination of said microfibrillar cellulose substrate.

20. The method of claim 15, including the step of selecting said source material from the group consisting of cotton, wood, flax and hemp.

21. The method of claim 15, including the step of sorting said particles by sieving the particles through screens for selecting particles of a desired size.

22. The method of claim 15, including the step of sorting said particles through an air classifying mill for selecting particles of a desired size.
